(19) United States
(12) Patent Application Publication
(10) Pub. No.: US 2008/0188907 A1
(43) Pub. Date: Aug. 7, 2008

(54) Controller for an Assistive Exoskeleton Based on Active Impedance

(75) Inventors: Gabriel Aguirre-Ollinger, Chicago, IL (US); Ambarish Goswami, Fremont, CA (US); J. Edward Colgate, Evanston, IL (US); Michael A. Peshkin, Evanston, IL (US)

Correspondence Address: HONDA/FENWICK, SILICON VALLEY CENTER, 801 CALIFORNIA STREET, MOUNTAIN VIEW, CA, 94041, US

(73) Assignees: Honda Motor Co., Ltd., Tokyo (JP); Northwestern University, Evanston, IL (US)

(21) Appl. No.: 11/696,110

(22) Filed: Apr. 3, 2007

Related U.S. Application Data
(60) Provisional application No. 60/888,035, filed on Feb. 2, 2007.

(51) Int. Cl. A61N 1/08

(52) U.S. Cl. 607/48; 601/5

(57) ABSTRACT

A system and method are presented to provide assist to a user by means of an exoskeleton with a controller capable of making the exoskeleton display active impedance. The exoskeleton assists the user by reducing the muscle effort required by the user to move his or her extremities. In one embodiment, a single-degree-of-freedom (1-DOF) exoskeleton assists a user with single-joint movement using an active impedance controller. In another embodiment, a multiple-degree-of-freedom (multi-DOF) exoskeleton assists a user with multiple-joint movement using an active impedance controller.

### CROSS-REFERENCE TO RELATED APPLICATION

This application claims priority from the following U.S. provisional patent application, which is hereby incorporated by reference: Ser. No. 60/888,035, filed on Feb. 2, 2007, entitled “Controller for an Assistive Exoskeleton Based on Active Impedance.”

### BACKGROUND OF THE INVENTION

1. Field of the Invention

The present invention relates to controlling an exoskeleton such that it can provide forces to assist a user's motion. One use of these forces is to reduce the muscular effort involved in ordinary motions of the lower extremities, such as walking, climbing stairs, sitting down and standing up. Said forces can also enhance the user's agility of movement. More particularly, the present invention relates to an innovative form of exoskeleton control based on producing a virtual modification of the mechanical properties of the user's extremities.

2. Description of Background Art

Most of the current implementations of assistive exoskeletons are still at the stage of research and development. Commercially available exoskeletons do not yet exist, although a number of groups are working towards them. Most of the existing designs function well only within a laboratory environment and require extensive adjustment and tuning by specialized personnel.

Exoskeleton designs can be classified in terms of their assistive capabilities as either passive or active devices. A passive device is one that cannot deliver more energy to the environment than it has previously drawn from the environment. Springs are an example of a simple passive mechanical device. Exoskeletons that display passive behavior thus have a limited assistive capability. Specifically, they can help the user employ his own muscle power more effectively, but they don't actually supply energy to the user. (In fact, they always draw a certain amount of energy from the user.)

One example of exoskeleton-based passive assist is passive gravity support where the exoskeleton supports part of the user's weight. However, the exoskeleton cannot contribute to raise the user's center of gravity, for example when getting up from a chair. A special case of gravity support is load-carrying assist, in which the exoskeleton supports part of a load carried by the user, for example a heavy backpack. Another passive assist is a force-offsetting assist, where the exoskeleton uses passive devices like springs to offset forces from one healthy body joint (such as the hip) to another body joint that is relatively weak due to some condition (such as the ankle in patients suffering from drop-foot gait). In a resonance-based assist, the exoskeleton modifies the dynamics of the limb to make it function closer to its resonant frequency, thus helping make more effective use of the user's own muscle power.

Active devices on the other hand behave as energy sources. Thus an active exoskeleton has the capability of supplying energy to the user in a continuous way. This is important because, in order to make an exoskeleton an all-purpose assistive device, it should be capable of active behavior. Human movements involve the elevation of the center of mass of the body at one point or another. Only an active device can assist this kind of motion in a repetitive way. Additionally, human motion involves a non-negligible amount of energy dissipation through muscle tissue. An active exoskeleton would provide the capability to supplement part of the energy dissipated by the human body.

Control of an exoskeletal device is a challenging problem. In the case of active exoskeletons, the prevailing paradigm is myoelectrical control. This control scheme consists of using the muscles' electromyographical (EMG) activity to estimate muscle forces and multiplying the estimated forces by a certain gain. Thus the exoskeleton behaves as an amplifier of muscle forces. This type of control has the appeal of being useful, in principle, to assist any motion attempted by the human. On the other hand, it has several practical limitations, due mainly to the nature of the EMG signal. Accurate estimation of torque from EMG is a challenging task requiring the characterization of several muscles, plus separating extraneous components affecting the EMG signal. Furthermore, EMG displays considerable variability with time and across subjects.

There is a need for an exoskeleton control method that eliminates the need for EMG as a source for the control signal, yet provides the versatility of assist that comes from active behavior.

### SUMMARY OF THE INVENTION

A system and method are presented to provide assist to a user by means of an exoskeleton with a controller capable of making the exoskeleton display active impedance. The exoskeleton assists the user by reducing the muscle effort required by the user to move his or her extremities.

In one embodiment, a single-degree-of-freedom (1-DOF) exoskeleton assists a user with single-joint movement using an active impedance controller. In another embodiment, a multiple-degree-of-freedom (multi-DOF) exoskeleton assists a user with multiple-joint movement using an active impedance controller.

### BRIEF DESCRIPTION OF THE DRAWINGS

The invention is illustrated by way of example, and not by way of limitation, in the figures of the accompanying drawings in which like reference numerals refer to similar elements.

FIG. 1 illustrates a schematic representation of a mechanical impedance, according to one embodiment.

FIG. 2 illustrates a 1-DOF assistive exoskeleton for a knee joint, according to one embodiment.

FIG. 3 illustrates a linear model of a 1-DOF exoskeleton, according to one embodiment.

FIG. 4 illustrates generating virtual impedance parameters in an exoskeleton through impedance control, according to one embodiment.

FIG. 5 illustrates a linear model of a human limb segment, according to one embodiment.

FIG. 6 illustrates a linear model of a system comprising a human limb segment attached to an exoskeleton, according to one embodiment.

FIG. 7 illustrates applying active exoskeleton impedance for scaling of a human limb impedance, according to one embodiment.

FIG. 8 illustrates the effect of pure negative damping on human limb impedance, according to one embodiment.

FIG. 9A illustrates an implementation of a 1-DOF assistive controller based on active admittance, according to one embodiment.

FIG. 9B illustrates an implementation of a 1-DOF assistive controller based on active impedance, according to one embodiment.

FIG. 10 illustrates an exoskeleton design with multiple degrees of freedom, according to one embodiment.

FIG. 11 illustrates human leg impedance parameters, according to one embodiment.

FIG. 12 illustrates the virtual modification of human leg dynamics through an exoskeleton assist, according to one embodiment.

FIG. 13 illustrates a control architecture for the multi-DOF exoskeleton, according to one embodiment.

### DETAILED DESCRIPTION OF THE EMBODIMENTS

A preferred embodiment of the present invention is now described with reference to the figures where like reference numbers indicate identical or functionally similar elements. Also in the figures, the left most digits of each reference number corresponds to the figure in which the reference number is first used.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the detailed description that follows are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “determining” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions described herein in the form of an algorithm. It should be noted that the process steps and instructions of the present invention could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the following claims.

1. Active Impedance

Mechanical impedance is the relationship between the net forces acting on a mechanical system and the system's resulting velocity or, for the case of rotational motion, the relationship between net torque and angular velocity.

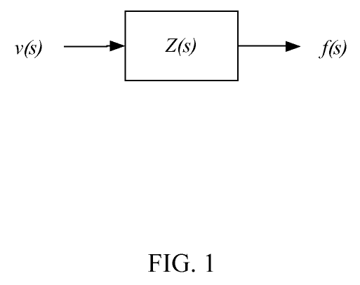

If the system is linear and time invariant, the impedance of the system can be expressed in compact form in the Laplace domain as the transfer function Z(s) relating the velocity v(s) to the net force f(s), as illustrated in FIG. 1. A second-order mechanical impedance is given by the expression

Z(s)=ms+b+(k/s) (1)

where the terms m, b and k represent, respectively, the mass, damping and stiffness (spring constant) of the system.

A physical system is said to be passive if the amount of energy ΔEₒᵤₜ that can be extracted from it over a certain period of time Δt is never greater than the sum of the system's initial energy Eₒ, plus the amount of energy ΔEᵢₙ that entered the system during Δt:

ΔEₒᵤₜ≦Eₒ+ΔEᵢₙ (2)

A passive impedance function Z(s) has no poles in the right half of the complex plane. In addition, any imaginary poles of Z(s) are simple, and have positive residues. Also, Re{Z(jω)}≧0 for all ω. An active system, by contrast, is not subject to the above conditions. Thus an active system is one that can behave as a continuous energy source.

The relationship between passive or active behavior and energy transfer can be understood in terms of phase. Suppose the input velocity to the system illustrated in FIG. 1 is sinusoidal, where v(t)=V sin ωt and V is a constant. The output force will show a phase shift φ relative to v(t) so that f(t)=F sin(ωt+φ) where F is a constant. It can be shown that the average power exchanged between the system and the environment is given by P=(FV/2)cos φ. A passive system is one in which the phase angle φ introduced by Z(s) has a value between −90° and +90°. In this case, the average power is positive, meaning that the system draws energy from the environment. The system will be active if the phase is either less than −90° or greater than +90°. In this case, the average power is negative, meaning that the system transmits energy to the environment.

Because of this phase angle condition, a passive system will possess positive impedance terms m, b and k. By contrast, an active system will possess a certain combination of negative impedance terms. Negative masses, dampers or springs do not normally occur in nature. Thus an active impedance cannot be generated from ordinary mechanical elements but requires an energy source plus control.

The control strategy focuses on enabling the exoskeleton to make the transition from passive to active behavior. During active behavior, the exoskeleton transmits energy to the user's limb. When the exoskeleton's behavior is passive, it draws energy from the user.

2. Assistive Control Based on Active Impedance: 1-DOF Case

The main concepts involved in assist through impedance control are explained using a simplified 1-DOF model of an exoskeleton. In one embodiment, this model is used to represent an exoskeleton designed to assist the motion of one of the leg's joints such as the knee joint. The desired assistive effect is to reduce the muscle torque at the joint by a given factor. Although the knee joint is used as an example, the exoskeleton and control strategy described herein can be used to assist any joint of any limb or extremity, such as the ankle, knee, or hip joint of a leg or the wrist, elbow, or shoulder joint of an arm. Also, the user can be a human or an animal. Additionally, for ease of explanation, the term “limb” may be used to describe a limb segment (such as a lower leg or an upper arm) attached to a joint of a limb.

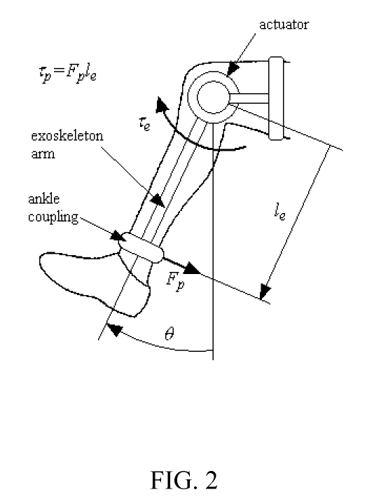

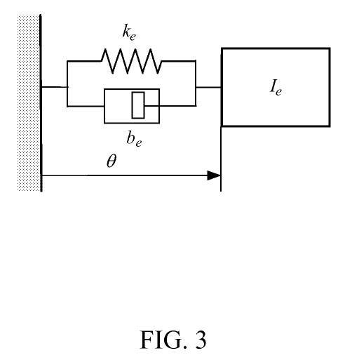

FIG. 2 illustrates a 1-DOF assistive exoskeleton for a knee joint, according to one embodiment. The illustrated exoskeleton includes an actuator and an arm and is coupled to the ankle. A linear model of a 1-DOF exoskeleton with its impedance parameters is illustrated in FIG. 3, according to one embodiment. The exoskeleton's impedance parameters include an inertia moment Iₑ (related to the exoskeleton's arm), a damping bₑ, and a stiffness kₑ. The “e” subscript refers to “exoskeleton.” Coordinate θ represents the angular position of the actuator of the exoskeleton.

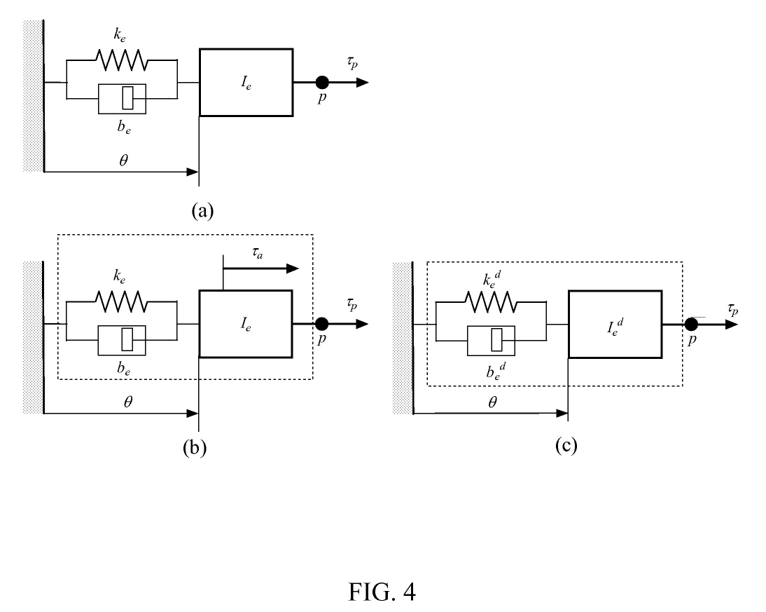

The terms Iₑ, bₑ, and kₑ represent real mechanical properties of the exoskeleton. Since these properties are passive and constant, they are of limited usefulness to the wearer. The exoskeleton's control and actuation modifies the dynamic behavior of the exoskeleton, replacing it with a set of virtual impedance parameters Iₑ^{d}, bₑ^{d}, kₑ^{d} that can serve to assist the human. The superscript “d” refers to “desired.” This form of control is referred to as impedance control, and it can be implemented through kinematic state feedback and/or force feedback. The concept is illustrated in FIG. 4, according to one embodiment.

In FIG. 4(a), the impedance felt by a user exerting a torque τₚ on the interaction port p of the exoskeleton is Zₑ(s)=Iₑs+bₑ+(kₑ/s), that is, the natural impedance of the exoskeleton. The “p” subscript refers to the “interaction port p,” a contact point between the user and the exoskeleton (e.g., the ankle coupling shown in FIG. 2). The interaction torque τₚ and interaction force Fₚ are also illustrated in FIG. 2. In FIG. 4(b), the exoskeleton is subject to an actuator torque τₐ in addition to the user's torque τₚ. The “a” subscript refers to the “actuator.” The torque τₐ is exerted by the actuator depicted in FIG. 2. The control law governing torque τₐ is such that the user feels now a different, virtual impedance of Zₑ^{d}(s)=Iₑ^{d}s+bₑ^{d}+(kₑ^{d}/s). This virtual impedance is illustrated in FIG. 4(c). From the user's perspective, the systems in FIGS. 4(b) and 4(c) have the exact same behavior.

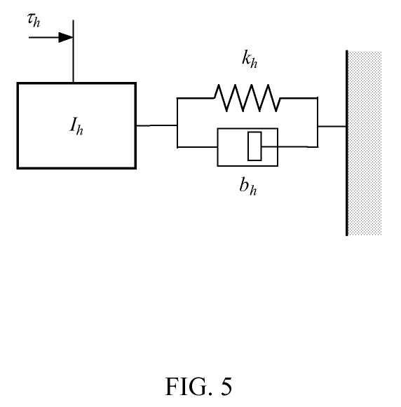

The interaction between the exoskeleton and the human user can be modeled in order to choose appropriate exoskeleton virtual impedance parameters that make the exoskeleton assistive to the user. FIG. 5 illustrates a linear model of the human limb segment, according to one embodiment. The limb's mechanical impedance comprises inertia moment Iₕ, damping bₕ, and stiffness kₕ. The “h” subscript refers to “human”. The human limb's impedance is described as Zₕ(s)=Iₕs+bₕ+(kₕ/s). When the exoskeleton is detached from the human limb, a certain muscle torque τₕ is needed in order to produce a desired velocity response ωₕ, per the following equation:

(Iₕs+bₕ+(kₕ/s))ωₕ=τₕ (3)

The exoskeleton is designed to reduce this needed muscle torque by a certain factor G greater than 1. In other words, with the exoskeleton assisting the limb, the user need provide a muscle torque of only τₕ*=τₕ/G to execute the same velocity ωₕ. The rest of the necessary torque is provided by the exoskeleton. Thus it is expected that the exoskeleton's virtual impedance Zₑ^{d}(s) is an active impedance. The virtual impedance Zₑ^{d}(s) can vary over time, for example if Zₕ(s) or G varies with time.

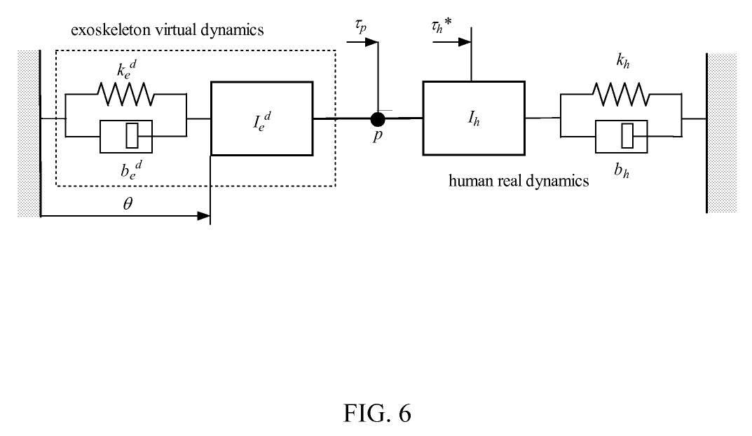

FIG. 6 illustrates a linear model of the coupled system formed by the exoskeleton's virtual dynamics and the human limb segment, according to one embodiment. τₚ represents the torque exerted by the exoskeleton on the human at the interaction port p. Assuming a rigid coupling between the exoskeleton and the human, the equations governing the motion of the exoskeleton and the human are

(Iₕs+bₕ+(kₕ/s))ωₕ=τₕ*−τₚ (4)

(Iₑ^{d}s+bₑ^{d}+(kₑ^{d}/s))ωₕ=τₚ (5)

To achieve the stated control objective of scaling the needed muscle torque per τₕ*=τₕ/G, we start by dividing each side of equation (3) by G:

(1/G)(Iₕs+bₕ+(kₕ/s))ωₕ=τₕ/G (6)

or

(1/G)(Iₕs+bₕ+(kₕ/s))ωₕ=τₕ* (7)

The virtual impedance parameters can be found by combining equations (4), (5) and (7). Thus the parameters are given by:

Iₑ^{d}s=Iₕ(1−G)/G (8)

bₑ^{d}=bₕ(1−G)/G (9)

kₑ^{d}=kₕ(1−G)/G (10)

The human impedance terms can be estimated by an appropriate method such as system identification based on least-squares approximation. As can be seen, the virtual impedance terms of the exoskeleton must be negative in order to achieve a scaling of the muscle torque. Given this condition, the passivity condition Re{Zₑ^{d}(jω)≧0 does not hold. In consequence, Zₑ^{d}(s) is an active impedance.

The exoskeleton scales down the human limb's impedance by adding to it a virtual exoskeleton impedance Zₑ^{d}(s) that is out of phase by exactly 180°. The modified human limb impedance is given by Zₕ^{d}(s):

Zₕ^{d}(s)=Zₕ(s)+Zₑ^{d}(s) (11)

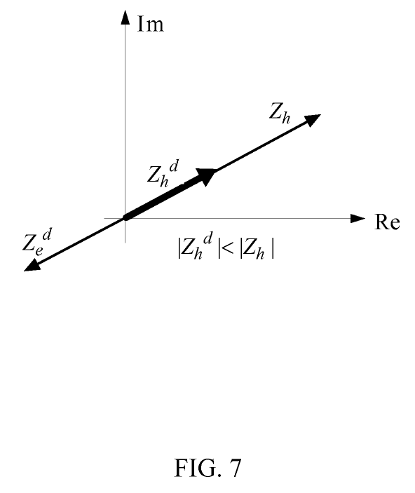

The summation of impedances in the above equation is illustrated in FIG. 7 where the impedances are represented as vectors in a complex plane.

Thus the assistive effect of the exoskeleton can be described as replacing the original human limb impedance Zₕ(s)=Iₕs+bₕ+(kₕ/s) by a “desired” human limb impedance Zₕ^{d}(s)=Iₕ^{d}s+bₕ^{d}+(kₕ^{d}/s), where the individual impedance terms are given by:

Iₕ^{d}=Iₕ+Iₑ^{d} (12)

bₕ^{d}=bₕ+bₑ^{d} (13)

kₕ^{d}=kₕ+kₑ^{d} (14)

The torque-scaling method just described is not the only possible way to apply active impedance for assistive purposes. In general, the exoskeleton will be assistive, in the sense of reducing the average muscle torque, if it can make the magnitude of the human's virtual impedance less than the magnitude of the human's natural impedance. Thus for the case of a sinusoidal (oscillating) limb motion of frequency c, the exoskeleton's action will be assistive if the following relationship holds:

|Zₕ^{d}(s)|_{s=jω}<|Zₕ(s)|_{s=jω} (15)

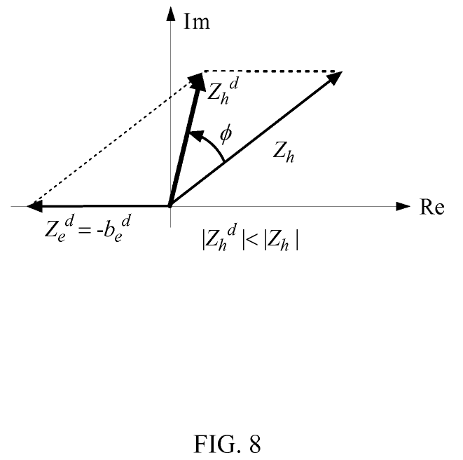

As an example of the previous statement, FIG. 8 illustrates the case in which a pure negative exoskeleton damping (Zₑ^{d}=bₑ^{d}, bₑ^{d}<0) is connected to the human limb. This would have the effect of canceling part of the normal energy dissipation that occurs in the limb due to the passive damping of the muscle tissue. As FIG. 8 illustrates, the magnitude of the modified human limb impedance, Zₕ^{d}, is less than that of the natural limb impedance, Zₕ.

3. Implementation of a 1-DOF Assistive Controller Based on Active Impedance

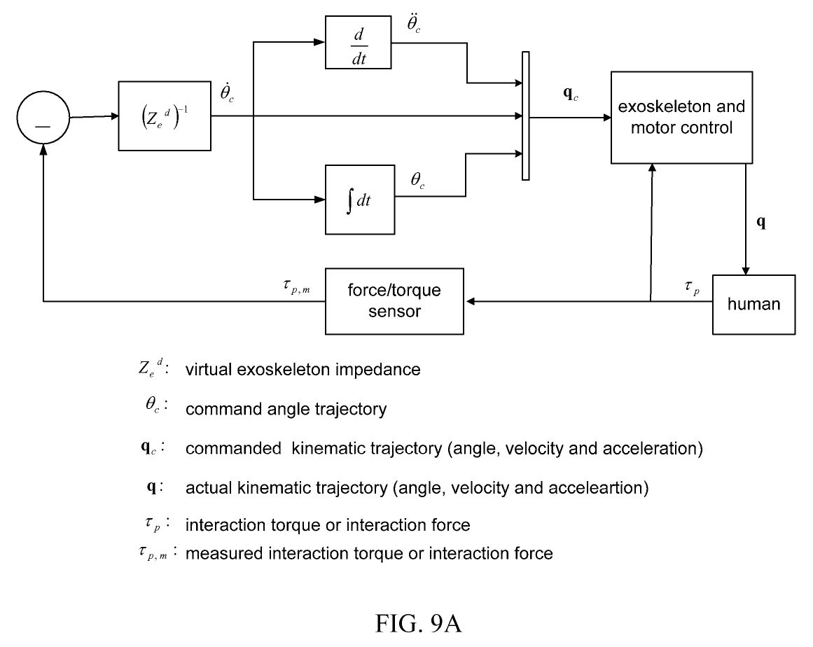

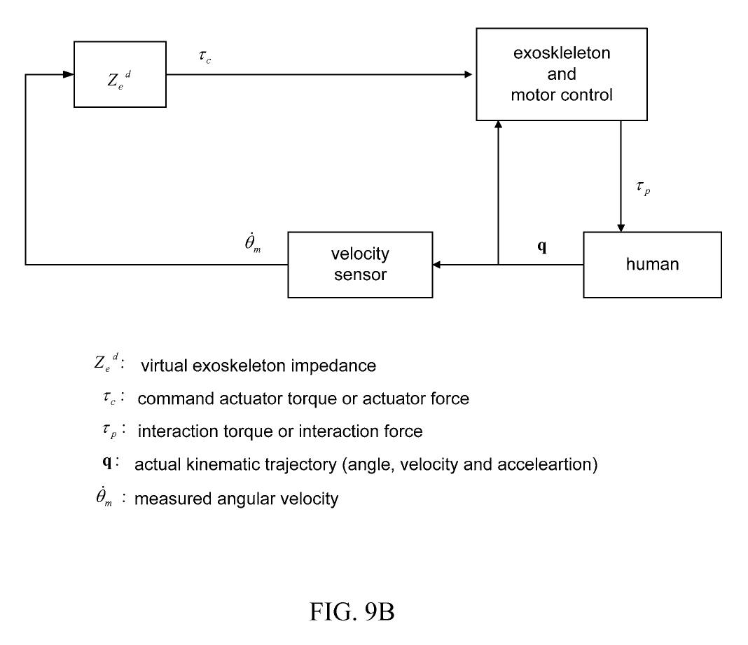

Implementing an active impedance controller can be accomplished in a number of ways. In one embodiment, the controller comprises an admittance-based trajectory command generator and a force/torque sensor in a feedback loop with the exoskeleton (including elements to control the actuator's motor) and the user. The structure of this controller is illustrated in FIG. 9A.

The measured interaction torque or interaction force τ_{p,m} is negated and used as input to an active impedance element containing the virtual impedance parameters of the exoskeleton in the form 1/Zₑ^{d}(s) (also known as virtual admittance). The active impedance element generates a commanded angle velocity \dot{θ}_{c}. The commanded angle velocity is possibly combined with its integral and/or derivative to form a commanded kinematic trajectory q_{c} (comprising angular position, angular velocity and/or angular acceleration) for the exoskeleton. The “c” subscript refers to “command” because the reference trajectory is commanded to the exoskeleton's motor. The commanded kinematic trajectory and the interaction torque or force τₚ is used to control the motor of the exoskeleton, possibly through a proportional-integral-derivative (PID) or other control mechanism. As a result of the exoskeleton's motor executing the command, the exoskeleton's actuator moves in a trajectory q (comprising angular position, angular velocity, and/or angular acceleration) and interacts with the human user to produce an interaction torque or force τₚ that is measured by a torque/force sensor to produce τ_{p,m} described above.

In another embodiment, an active impedance controller comprises an impedance-based torque command generator and a velocity sensor in a feedback loop with the exoskeleton (including elements to control the actuator's motor) and the user. The structure of this controller is illustrated in FIG. 9B.

The measured angular velocity \dot{θ}ₘ is used as an input to an active impedance element containing the virtual impedance parameters of the exoskeleton in the form Zₑ^{d}(s). The active impedance element generates a commanded actuator torque or force τ_{c}. The commanded actuator torque or force τ_{c} and the actual kinematic trajectory of the joint of the human user q (comprising angular position, angular velocity, and/or angular acceleration) is used to control the motor of the exoskeleton, possibly through a proportional-integral-derivative (PID) or other control mechanism. As a result of the exoskeleton's motor executing the command, the exoskeleton exerts an interaction torque or interaction force τₚ on the user and the user's joint moves in the kinematic trajectory q which is measured by the velocity sensor to produce \dot{θ}ₘ described above.

The active impedance controller is capable of (on average) inputting energy to the user-exoskeleton system. The power exerted by the exoskeleton is the product of the exoskeleton angular velocity (the velocity component of q) and interaction torque (τₚ). The integral of this power is the energy input by the exoskeleton to the system, which is positive on average.

The active impedance controller can be implemented in hardware, software, or both. The signals in the controller may be digital, analog, or both. The modules illustrated in FIGS. 9A and 9B can be combined or further divided into other modules. The impedance controller can be implemented as a part of the exoskeleton, as a part of the exoskeleton's actuator, or separate from the exoskeleton.

4. Assistive Control Based on Active Impedance: Multi-DOF Case

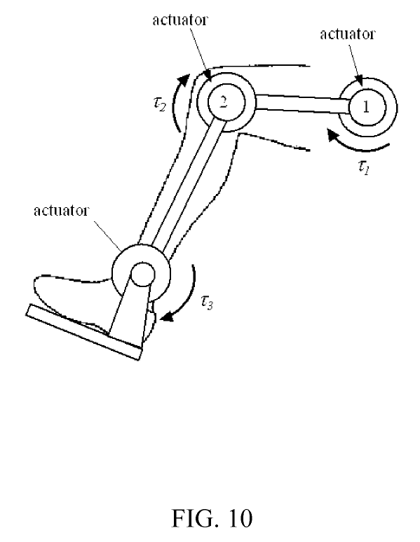

FIG. 10 illustrates an exoskeleton design with multiple degrees of freedom (multi-DOF), according to one embodiment. The exoskeleton is composed of rigid links connected by movable joints with actuation. Such a device can be used to assist the user in performing the more complex lower-limb motions associated with daily living such as walking, climbing stairs, sitting down, etc. The exoskeleton illustrated in FIG. 10 is a nonlinear system with multiple inputs, τ₁, τ₂, and τ₃, which are the actuators' torques, and multiple outputs, which are the velocity responses of the links. The control methods described in the previous sections can be extended to the multi-DOF exoskeleton.

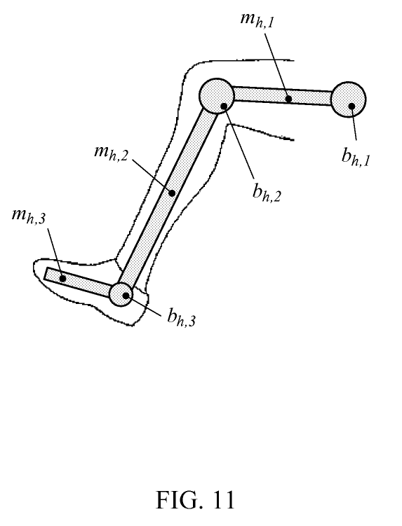

FIG. 11 illustrates the impedance properties of the human limb, according to one embodiment. The leg is represented as a multi-link mechanism characterized by the mass of each link, m_{h,i} (on which the moment of inertia also depends). The link masses can be arranged into a mass vector mₕ. Each joint is characterized by a damping coefficient, b_{h,i}. The damping coefficients can be arranged into a damping matrix Bₕ. (Joint stiffnesses have been left out for clarity.) The exoskeleton is designed to produce a virtual modification of the impedance parameters of the limb, thus reducing the muscle effort needed to move the limb. The modified impedances are represented as a set of virtual masses, mₕ^{d} and damping coefficients, Bₕ^{d}. Assist can be achieved by making each of the virtual masses mₕ^{d} smaller than its real counterpart in mₕ. The effect perceived by the user would be the limb weighing less and showing less inertia. Similarly, a virtual reduction in the damping of the joints can be expected to have an assistive effect on the user.

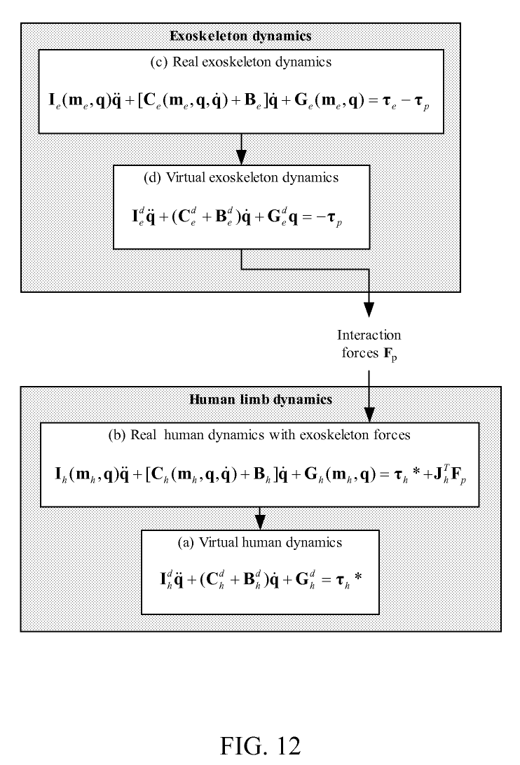

FIG. 12 illustrates, in schematic form, one way in which the controller for the multi-DOF exoskeleton produces the virtual modification in the properties of the human limb. The natural dynamics of the human limb are represented by the following equation:

Iₕ(mₕ,q)\ddot{q}+[Cₕ(mₕ,q,\dot{q})+Bₕ]\dot{q}+Gₕ(mₕ,q)=τₕ (16)

In the above equation, vector q is a set of n generalized coordinates (typically joint angles) representing the configuration of the limb in joint space. Iₕ (mₕ,q) is the inertia matrix of the limb, Cₕ (q,\dot{q}) represents the centrifugal and Coriolis terms, and Gₕ (mₕ,q) represents the gravitational forces acting on the limb. Bₕ is the damping matrix of the limb, and the vector τₕ represents the net muscle torques acting on the limb's joints. The effect of the exoskeleton is replacing the limb's natural dynamics by a set of virtual dynamic terms denoted by the superscript d in block (a) of FIG. 12:

Iₕ^{d}\ddot{q}+(Cₕ^{d}+Bₕ^{d})\dot{q}+Gₕ^{d}=τₕ* (17)

where

Iₕ^{d}=Iₕ(mₕ^{d},q) (18)

Cₕ^{d}=Cₕ(mₕ^{d},\dot{q}) (19)

Gₕ^{d}=Gₕ(mₕ^{d},q) (20)

One way to produce the virtual impedance of the limb is through the interaction forces Fₚ (in Cartesian space) between the exoskeleton and the human limb. These forces modify Equation 16 as shown in block (b) of FIG. 12.

Iₕ(mₕ,q)\ddot{q}+[Cₕ(mₕ, q,\dot{q})+Bₕ]\dot{q}+G ₕ(mₕ,q)=τₕ*+Jₕ^{T}Fₚ (21)

In this equation, Jₕ^{T} is the Jacobian matrix of the human limb. The Jacobian matrix relates the Cartesian velocities \dot{x} of the points where the forces Fₚ are applied, to the limb joints' angular velocities \dot{q}. The last term in the above equation can be replaced by a vector of equivalent torques τₚ in joint space:

τₚ=Jₕ^{T}Fₚ (22)

These torques can be measured directly, for instance, by installing torque sensors at the joints. Combining Equations 17, 21, and 22 yields the following equation (with some mass and state terms removed for clarity):

(Iₕ^{d}−Iₕ)\ddot{q}+[(Cₕ^{d}−Cₕ)+(Bₕ^{d}−Bₕ)]\dot{q}+(Gₕ^{d}−Gₕ)q=−τₚ (23)

This equation can be expressed in compact form by defining

Iₑ^{d}=Iₕ^{d}−Iₕ, Cₑ^{d}=Cₕ^{d}−Cₕ, Bₑ^{d}=Bₕ^{d}−Bₕ, Gₑ^{d}=Gₕ^{d}−Gₕ (24)

We refer to the above terms as the virtual dynamics of the exoskeleton. Thus,

Iₑ^{d}\ddot{q}+(Cₑ^{d}+Bₑ^{d})\dot{q}+Gₑ^{d}q=−τₚ (25)

As expected, the virtual dynamics of the exoskeleton are those of an active system. For the particular case of a virtual damping matrix Bₑ^{d} composed of constant terms, the virtual dynamics of the exoskeleton will be active if Bₑ^{d} is proven to be negative definite.

Equation 25, shown also in block (d) of FIG. 12, represents the basic control law for the exoskeleton. As in the case of the 1-DOF exoskeleton, one embodiment of this control law is an impedance controller. In such an impedance controller, given the interaction torque −τₚ as input, the exoskeleton enforces the kinematic trajectory represented by \ddot{q}, \dot{q} and q.

Equation 25 does not represent the real dynamics of the exoskeleton. These are represented instead by the equation in block (c) of FIG. 12:

Iₑ(mₑ,q)\ddot{q}+[Cₑ(mₑ,q,\dot{q})+Bₑ]\dot{q}+Gₑ(mₑ,q)=τₑ−τₚ (26)

In the above equation, Iₑ (mₑ,q) is the inertia matrix of the exoskeleton, Cₑ (q,\dot{q}) represents the centrifugal and Coriolis terms, and Gₕ (mₕ,q) represents the gravitational forces acting on the exoskeleton. Bₑ is the damping matrix of the exoskeleton. Vector τₑ represents the actuators' torques. The controller's task is to replace these dynamics with those from Equation 25. This normally involves the use of state and/or force feedback.

5. Implementation of a Multi-DOF Assistive Controller Based on Active Impedance

One consideration in implementing a control architecture for a multi-DOF exoskeleton is the linearization of the exoskeleton plant, that is, making the dynamic properties of the exoskeleton independent of the inputs to the system. As shown below, linearization can be accomplished through the use of a model of the dynamics of the physical exoskeleton.

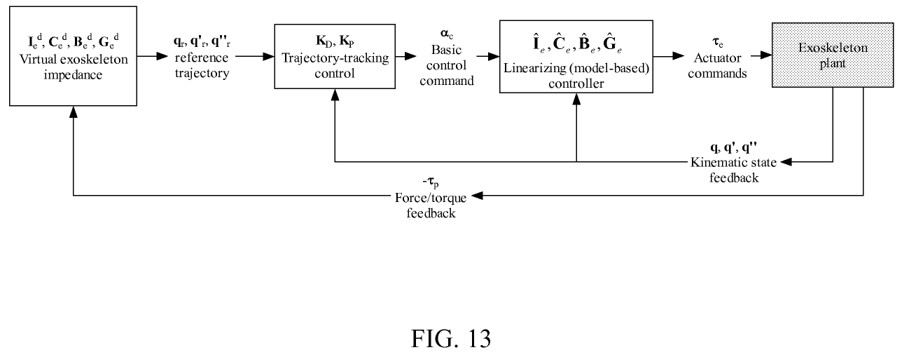

FIG. 13 illustrates a diagram of the control architecture for the multi-DOF exoskeleton, in one embodiment. The exoskeleton's control comprises three main stages, each of which has its own feedback loop. The first stage is the active impedance element based on the virtual exoskeleton impedance. This element represents the desired dynamic behavior of the exoskeleton. The output of the active impedance element is a reference kinematic trajectory (comprising angular position, angular velocity, and/or angular acceleration) for each of the exoskeleton's actuators. The second stage is the trajectory-tracking controller. This component has the function of issuing the basic control commands necessary for the actuators to follow the reference trajectory. This control block can contain a proportional (P) or proportional-derivative (PD) controller. The third stage is the linearizing (model-based) controller. In the case of a multi-DOF exoskeleton, gravity and coupling between the links are sources of nonlinear dynamics that make the trajectory-tracking control insufficient. This problem is solved by adding a linearizing control that effectively makes the exoskeleton behave as a linear plant. This control stage combines a model of the exoskeleton's true dynamics with kinematic feedback (typically position and velocity) from the physical exoskeleton.

The controller illustrated in FIG. 13 is designed to perform the task outlined in FIG. 12. In one embodiment, the first control stage comprises an active impedance element based on equation 25. This element receives the measured interaction torque −τₚ and generates a reference acceleration trajectory \ddot{q}ᵣ. Successive integrations of this term generate a reference velocity \dot{q}ᵣ and a reference position qᵣ.

The second stage is the trajectory-tracking controller (outer-loop control), for example a PD controller that applies the control law

α_{c}=\ddot{q}ᵣ+K_{D}ėᵣ+K_{P}eᵣ (27)

where ėᵣ and eᵣ are, respectively, the velocity error and the position error. K_{D} and K_{P} are scalar gain matrices. α_{c} is the commanded acceleration input to the exoskeleton.

The third stage is a model-based controller that translates the commanded acceleration into torque commands τₑ for the actuators. Linearization of the exoskeleton also takes place at this stage. On the basis of Equation 26, the control law for the third stage is given by

τₑ=Îₑ(mₑ,q)\ddot{q}+[Ĉₑ(mₑ,q,\dot{q})+\hat{B}ₑ]\dot{q}+Ĝₑ(mₑ,q) (28)

The terms Îₑ(q), Ĉₑ (q,\dot{q}), \hat{B}ₑ and Ĝₑ(q) constitute the model of the exoskeleton's real dynamics. Provided that the model terms are sufficiently accurate, the third stage of the controller yields a cancellation of the nonlinear terms in Equation 26. The resulting behavior of the physical exoskeleton is thus given by

\ddot{q}=α_{c}−Iₑ(mₑ,q)⁻¹τₚ (29)

The above equation shows that the exoskeleton's kinematic response \ddot{q} is linear to the inputs α_{c} (control) and −τₚ (interaction torque).

Although the invention has been described in considerable detail with reference to certain embodiments thereof, other embodiments are possible as will be understood to those skilled in the art.

What is claimed is:

1. A system for controlling an actuator of an exoskeleton, comprising:
an active impedance element configured to output a kinematic trajectory command for the actuator based on a measured interaction torque or force, wherein the measured interaction torque or force represents an interaction between the exoskeleton and a limb segment of a user wearing the exoskeleton.

2. The system of claim 1 wherein the active impedance element is configured to be assistive to the user wearing the exoskeleton by reducing a muscle torque required to move the limb segment at a particular angular velocity.

3. The system of claim 1 wherein the active impedance element is configured to decrease a magnitude of a natural impedance of the limb segment when the exoskeleton is coupled to the limb segment.

4. The system of claim 1, wherein the active impedance element has an impedance equal to Zₑ^{d}(jω), wherein Zₑ^{d}(jω) represents an active impedance of the exoskeleton.

5. The system of claim 1, wherein the active impedance element is configured to decrease one or more natural mechanical impedance parameters of the limb segment when the exoskeleton is coupled to the limb segment, the parameters comprising an inertia moment Iₕ, a damping bₕ, and a stiffness kₕ.

6. The system of claim 5, wherein the active impedance element is configured so that one or more virtual impedance parameters of the exoskeleton are negative, the parameters comprising an inertia moment Iₑ^{d}, a damping bₑ^{d}, and a stiffness kₑ^{d}.

7. The system of claim 6, wherein the active impedance element is configured to be assistive to the user wearing the exoskeleton by reducing a muscle torque required to move the limb segment at a particular angular velocity, wherein the required muscle torque is reduced by a factor of G greater than 1, and wherein the virtual impedance parameters of the exoskeleton are chosen so that Iₑ^{d}=Iₕ(1−G)/G, bₑ^{d}=bₕ(1−G)/G, and kₑ^{d}=kₕ(1−G)/G.

8. The system of claim 1, further comprising:
a feedback loop configured to decrease a trajectory error exhibited by the exoskeleton.

9. The system of claim 8, wherein the trajectory error is a difference between the trajectory command and a trajectory exhibited by the exoskeleton.

10. A system for controlling an actuator of an exoskeleton, comprising:
an active impedance element configured to output a torque or force command for the actuator based on a measured angular velocity of a limb segment of a user wearing the exoskeleton.

11. The system of claim 10 wherein the active impedance element is configured to be assistive to the user wearing the exoskeleton by reducing a muscle torque required to move the limb segment at a particular angular velocity.

12. The system of claim 10 wherein the active impedance element is configured to decrease a magnitude of a natural impedance of the limb segment when the exoskeleton is coupled to the limb segment.

13. The system of claim 10, wherein the active impedance element has an impedance equal to Zₑ^{d}(jω), wherein Zₑ^{d}(jω) represents an active impedance of the exoskeleton.

14. The system of claim 10, wherein the active impedance element is configured to decrease one or more natural mechanical impedance parameters of the limb segment when the exoskeleton is coupled to the limb segment, the parameters comprising an inertia moment Iₕ, a damping bₕ, and a stiffness kₕ.

15. The system of claim 14, wherein the active impedance element is configured so that one or more virtual impedance parameters of the exoskeleton are negative, the parameters comprising an inertia moment Iₑ^{d}, a damping bₑ^{d}, and a stiffness kₑ^{d}.

16. The system of claim 15, wherein the active impedance element is configured to be assistive to the user wearing the exoskeleton by reducing a muscle torque required to move the limb segment at a particular angular velocity, wherein the required muscle torque is reduced by a factor of G greater than 1, and wherein the virtual impedance parameters of the exoskeleton are chosen so that Iₑ^{d}=Iₕ(1−G)/G, bₑ^{d}=bₕ(1−G)/G, and kₑ^{d}=kₕ(1−G)/G.

17. The system of claim 10, further comprising:
a feedback loop configured to decrease a torque error or force error.

18. The system of claim 17, wherein the torque error or force error is a difference between the torque command or force command and a measured interaction torque or force, wherein the measured interaction torque or force represents an interaction between the exoskeleton and a limb segment of a user wearing the exoskeleton.

19. A system for controlling an actuator of an exoskeleton, comprising:
an active impedance element capable of inputting energy, on average, to a system comprising a limb segment of a user and the exoskeleton, wherein the active impedance element transfers energy, on average, from the actuator to the limb segment by exerting an interaction torque or force on the limb segment, and wherein the active impedance element transfers energy to the limb segment only in response to movement of the limb segment, the movement producing an interaction torque or force with the exoskeleton.

20. A system for controlling a plurality of actuators of an exoskeleton, comprising:
an active impedance element configured to output a set of kinematic trajectory commands, one command for each actuator, based on a measured interaction torque or force vector, wherein the measured interaction torque or force vector represents interactions between the exoskeleton and a limb segment of a user wearing the exoskeleton.

21. The system of claim 20, further comprising:
a trajectory-tracking controller configured to output a set of first control commands for the actuators to follow the reference kinematic trajectory, wherein the first control commands comprise an acceleration;
a linearizing controller that enables the exoskeleton to behave as a linear plant, the linearizing controller configured to output a set of second commands, wherein the second commands comprise torque commands for the actuators; and
an exoskeleton plant that includes a system comprising the exoskeleton and the limb segment, wherein the exoskeleton plant outputs to the active impedance element either force feedback or torque feedback and outputs to the trajectory-tracking controller and the linearizing controller kinematic state feedback, wherein the kinematic state feedback comprises one alternative of a group containing a position, a velocity, and an acceleration.
